(19) United States
(12) Patent Application Publication
(10) Pub. No.: US 2008/0038760 A1
(43) Pub. Date: Feb. 14, 2008

(54) Method for Detecting Anti-Transglutaminase Antibodies

(75) Inventors: Francoise Mascart, Bruxelles (BE); Annick Ocmant, Le Roeulx (BE)

Correspondence Address: KNOBBE MARTENS OLSON & BEAR LLP, 2040 MAIN STREET, FOURTEENTH FLOOR, IRVINE, CA, 92614, US

(73) Assignee: Method for Decting Anti-Transglutaminase Antibodies

(21) Appl. No.: 11/629,030

(22) PCT Filed: Jun. 6, 2005
(86) PCT No.: PCT/EP05/06088

§ 371 Date: Oct. 18, 2007

(30) Foreign Application Priority Data
Jun. 8, 2004 (EP) EP2004006174

(51) Int. Cl. G01N 33/00

(52) U.S. Cl. 435/7.92

(57) ABSTRACT

Methods for detecting anti-transglutaminase antibodies in a saliva sample containing the antibodies are disclosed. Saliva samples are pre-treated. The antibodies are subsequently detected in the pre-treated saliva by an immune reaction with a transglutaminase under conditions suitable for forming immuno-complexes with the antibodies. The method is useful for diagnosing and/or therapeutically controlling celiac disease.

### FIELD OF THE INVENTION

The invention relates to a method for detecting anti-transglutaminase antibodies in saliva for diagnosis or therapeutic control of the coeliac disease.

### TECHNOLOGICAL BACKGROUND OF THE INVENTION

The coeliac disease is a gluten induced autoimmune enteropathy in genetically susceptible subjects. Gluten is the endosperm protein fraction of wheat, rye, barley and oat. Wheat flour, made from the endosperm, contains various proteins among which gliadin, a protein of the prolamin family that is the alimentary antigen responsible of such disease.

On one hand, there are genetic factors predisposing to the coeliac disease, indeed, the H1a genes contribution is well shown, and on the other hand one or more environmental inducing factors of undetermined origin.

The active phase of the disease is associated with the production of antigliadin antibodies and of autoantibodies, the anti-endomysium antibodies. These antibodies are directed against the connective tissue which surrounds the smooth muscle fibers. The antigen recognized by the anti-endomysium antibodies is an enzyme, the tissue transglutaminase, recently discovered by the Dieterich's team. This enzyme seems to be involved in the disease pathogenesis: the transglutaminase catalyses the covalent binding between a lysine residue and a glutamine residue of the proteins. Therefore, it was suggested that the binding of transglutaminase to gliadin or to some gliadin peptides induces the generation of neo-antigens that are recognized by the immune system and thus the production of autoantibodies against transglutaminase.

Under its classical form, the coeliac disease is characterized by either a total or subtotal villous atrophy predominant at the level of the proximal small intestine and secondary to the ingestion of gluten.

The infant or young child shows a chronic diarrhea with most often as well as a slow growth and a ballooned abdomen. More seldom, as to the older child, the disease can be less typical, limited to an isolated small size, a chronic iron deficiency anemia, abnormalities of the dental enamel or arthralgia.

In adults, the disease symptoms adults are very variable. Current signs are, as for child, diarrhea and a worrying loss of weight. More often than for latter, the disease is either mono-symptomatic (iron deficiency anemia, osteoporosis . . . ) or atypical (expressed by muscular cramps, aphthous stomatitis, menstrual irregularities even repeated spontaneous abortions, break-down).

Besides the classical forms of the disease which represent only a minority of cases, numerous are the patients who, in the absence of major clinical signs, show an extended villous atrophy of the jejunal mucosa. These pauci-symptomatic forms of the coeliac disease clinically appear most often under the form of an anemia due to iron and/or folic acid deficiency or under the form of an osteoporosis or an osteomalacia.

Some people present a higher susceptibility to coeliac disease: family members of sick individuals, patients with dermatitis herpetiformis, with insulin-dependent diabetes, an autoimmune chronic thyroiditis or a selective deficiency in IgA. Silent forms of the coeliac disease are particularly predominant in these groups.

The early detection of the coeliac disease defaced or subclinical forms is therefore difficult, but however important in order to, in the one hand, avoid the complication linked to a nutrition gap secondary to the malabsorption and in the other hand, the development of lymphomas or digestive carcinomas which might form in 15% of non-treated patients.

The coeliac disease is especially seen in caucasian and more frequently in woman. Generally, the disease appears in early childhood. The prevalence of the coeliac disease in Europe, comprised between 1/1000 and 1/2000 inhabitants, could be in fact higher if the pauci-symptomatic and silent forms of the disease were to be taken into account, which would bring the disease prevalence between 1/200 and 1/400.

The detection of two types of serum (humoral) antibodies, the anti-endomysium antibodies on the one hand and the anti-gliadin antibodies on the other hand, presently constitutes a good screening test for coeliac disease. The IgA anti-endomysium represents the most reliable marker for coeliac disease detection. The sensitivity of such test is above 90%, and the specificity thereof approaches 100%. These antibodies are searched by an indirect immunofluorescence technique on slices of primate esophagus or slices of human umbilical cords. The presence of antigliadin antibodies in the serum of patients with coeliac disease is long known, the detection thereof being done by immunoassays, however the lack of specificity of such test provides a diagnosis tool of little reliability when used on its own. The anti-gliadin IgG screening is a sensitive but hardly specific test, while that of IgA is more specific but less sensitive, in view of the higher frequency of IgA deficiency in patients with coeliac disease.

There exist also immunoassays that allow the detection of anti-transglutaminase IgA antibodies in the serum. These tests make use of a transglutaminase either from guinea pig kidney or human recombinant transglutaminase. The latter provide a specificity that may be compared to that of anti-endomysium.

The only element for a positive diagnosis lays on the intestinal mucosa's histology. The intestinal impairment is located at the level of the proximal small intestine and is typically associated with either a subtotal or total villous atrophy, a hypertrophy of the crypts, a massive lymphocytic infiltration of the epithelium and a lymphoplasmacytic infiltrate of the chorion, all of these elements being responsible for the malabsorption syndrome.

The treatment of patients with coeliac disease is based on a strictly free gluten diet, a diet which is particularly difficult to follow. When establishing the diet, the antibodies characteristic of the coeliac disease disappear from the serum in a progressive manner, thus providing an evaluation of the degree of adhesion to the diet. However, here again a blood collection is required.

Screening for the presence of anti-endomysium IgAs or anti-transglutaminase IgAs in blood provides a most precious aid for the diagnosis of gluten intolerance which in Europe concerns about one individual out of 300. However, in order to avoid the blood collection requirement, it would be of interest to have a test that to could be conducted on saliva samples.

Unfortunately, when previously such a test was applied by means of an ELISA type process onto saliva samples, the results obtained did not allows differentiating gluten intolerant patients from those who are not. The recently disclosed RIA process is restricting its application to laboratories that make use of radioactive products.

The aim of the present invention is to provide a non-invasive solution for diagnosis and monitoring the coeliac disease through a cost effective and efficient diagnostic test capable to overcome, at least partly, the drawbacks of the solutions known from the prior art.

### BRIEF SUMMARY OF THE INVENTION

The above aim is achieved by the invention as defined by the set of appended claims.

Therefore, the aim of the present invention is to provide a method for detecting and/or dosing anti-transglutaminase antibodies in a saliva sample susceptible to contain said antibodies, characterized in that the saliva is collected in a saliva collector so as to avoid a degradation of the antibodies, subsequently the saliva sample undergoes a pre-treatment, then said antibodies are detected in the pre-treated saliva by an immunoreaction with a transglutaminase under conditions suitable to the formation of immuno-complexes with said antibodies.

In a particular embodiment, the present invention comprises non radioactive immuno-enzymatic process efficient for the screening of anti-transglutaminase IgA in saliva including pre-treating the salivas with a solution containing a mucolytic compound, preferably the N-acetyl-cystein, after having collected them in a convenient manner. The samples are collected in such a way as to avoid antibody degradation.

According to a particular embodiment of the invention, the method comprises collecting the salivas in a sampling device and pre-treating the sampled salivas which includes bringing the saliva in contact with a mucolytic compound. The compound may be selected from the group comprising N-acetyl-cystein, nacystelyn, dithiothreitol, gelsolin, thioredoxin and EDTA.

The aim is to treat the salivas with a mucolytic compound, preferably the N-acetylcystein in order to degrade the chemical bonds between the IgA molecules and the mucins present in saliva. This treatment permits to free the IgA from the mucins, thus avoiding a conjonctive non specific sticking of the mucins to the microtitration plates and increasing the detectability of the anti-transglutaminase IgA.

Such pre-treatment allows to decrease the non specific binding of samples not containing anti-transglutaminase antibodies and to increase the signal from samples containing anti-transglutaminase antibodies thus allowing a marked improvement of the discrimination between both positive and negative salivas as to the presence of anti-transglutaminase IgA.

By allowing only the binding of specific IgAs to a plate coated with a transglutaminase and by improving the anti-transglutaminase IgA detection, a better discrimination is achieved between the saliva of gluten intolerant individuals and that of individuals which are not.

A preliminary step of the method according to the invention consists not solely to collect the salivas in special tubes intended to such use (sampling device), but also to apply to the salivas a pre-treatment before the screening for antibodies by means of an immuno-enzymatic technique.

According to a particular embodiment of the invention, the saliva sampling device comprises: a sample collector which receives selectively a saliva sample; and an indicator activated by means of a pre-selected quantity of saliva sample received in order to indicate that the quantity of collected biological fluid sample is adequate. According to a preferred embodiment of the invention, the method is characterized in that it comprises collecting the salivas in a sampling device as disclosed in the documents WO95/02996, U.S. Pat. No. 5,260,031, such as Omni-SAL®, in the documents WO97/24979, WO96/04850, WO95/27205 such as OraSure®, in the document U.S. Pat. No. 4,774,9§2 such as Salivette®. Preferably, the sampling device is selected from the group comprising: Omni-SAL® (Saliva Diagnosis Systems, Inc.), Salivette®(Sarstedt Ltd.), Orapette® (Trinity Biotech) and OraSure@) (Epitope, Inc.), preferably Omni-Sal®.

Other specificities and advantages of the present invention will be apparent on reading the following disclosure and examples which are given for illustration.

### BRIEF DISCLOSURE OF THE FIGURES

FIG. 1 represents a diagram depicting the anti-transglutaminase ELISA concept according to a particular embodiment of the invention.

FIG. 2 represents a graph comparing various N-acetylcystein concentrations according to a particular embodiment of the invention.

FIG. 3 represents a graph comparing non treated salivas and salivas treated with N-acetylcystein according to a particular embodiment of the invention.

FIG. 4 represents a graph comparing non treated salivas and salivas treated with N-acetyl-cystein according to a particular embodiment of the invention with TG from Guinea pig kidney.

FIGS. 5 and 6 represent graphs comparing non treated salivas and salivas treated with N-acetyl-cystein according to particular embodiments of the invention with a pre-existing kit for anti-TG antibody dosage in serum (Celikey, Pharmacia Diagnostics).

FIGS. 7 and 8 represent graphs comparing the level of anti-TG IgA in non treated salivas and salivas treated with N-acetyl-cystein from healthy control subjects (FIG. 7) and untreated patients with coeliac disease (FIG. 8) according to particular embodiments of the invention.

FIG. 9 represents a graph comparing the level of anti-TG IgA in non treated salivas and salivas treated with N-acetyl-cystein from healthy control subjects and untreated patients with coeliac disease according to a particular embodiment of the invention.

FIG. 10 represents a graph comparing the level of anti-TG IgA in salivas treated with N-acetyl-cystein at the level of serum anti-TG IgA according to a particular embodiment of the invention.

FIG. 11 represents a graph comparing the level of anti-TG IgA in salivas treated with N-acetyl-cystein from healthy control subjects, from pathologic control subjects and from untreated patients with coeliac disease according to a particular embodiment of the invention.

FIG. 12 represents a graph comparing the level of anti-TG IgA in salivas treated with N-acetyl-cystein from healthy control subjects, from untreated patients with coeliac disease and from coeliac patients under diet according to a particular embodiment of the invention.

### DETAILED DESCRIPTION OF THE INVENTION

The method of the invention uses the human saliva as a non invasive source, in substitution to invasive serum or blood, for detecting and/or dosing anti-transglutaminase antibodies.

The IgA are immunoglobulins that are prevalent in the secretions wherein they play an important role in protecting the organism against external attacks. Most of the IgA to be found in the saliva is present under a dimeric form: the secretory IgA or sIgA, constituted of two monomeric IgAs linked by a small molecule called the “J chain” and a secretory glycoprotein called the Secretory Componant. The IgA which is kept under the form of a dimer by the J chain is secreted by the under-epithelial plasmocytes; it binds to the secretory component and subsequently crosses the epithelial barrier. The secretory component assists the transport of IgA through the secretions and protects it from proteolysis.

The detection of the coeliac disease in patients with pauci-symptomatic or silent forms of coeliac disease is often difficult and delayed. Nevertheless, these patients experiment an improvement of their overall condition when they start a diet.

Thus, a large scale screening among a large population is meaningful, although it may be devised only with the use of an almost no invasive tests. The present invention allows dosing the anti-transglutaminase IgA antibodies using the ELISA process on salivary samplings.

The present invention relates to a method for detecting anti-transglutaminase antibodies in a saliva sample susceptible to contain said antibodies, characterized in that the saliva sample undergoes a pre-treatment. In a preferred embodiment the method comprises on the one hand the collection of the saliva in a sampling device such as Omni-SAL® salivettes and on the other hand the pre-treatment which consists in bringing the saliva in contact with a mucolytic compound such as N-acetyl cystein.

The transglutaminase used according to the invention may be of human, animal, synthetic or recombinant origin.

The detection may be performed in an immuno-assay, for example by direct or indirect coupling with a reaction partner containing a marking substance easy to detect.

According to a preferred embodiment, the detection is performed in an ELISA test. For example, the formed immuno-complexes can be brought in contact with a conjugate constituted of a labeled antibody directed against said antibodies, under conditions suitable to the formation of labeled immuno-complexes, subsequently the labeled immuno-complexes are detected and quantified. For example the conjugate may be an anti-immunoglobulin antibody labeled with alkaline phosphatase or with peroxidase, subsequently the formation of the labeled immuno-complexes is detected and quantified by colorimetry or fluorometry.

The present method allows the detection of gluten induced diseases such as the coeliac disease. Moreover, the present method allows monitoring a gluten free diet during which normally the antibodies should disappear.

Furthermore, the present invention relates to a diagnostic method or a method for the therapeutic monitoring of the coeliac disease, characterized in that anti-transglutaminase antibodies are detected in a saliva sample susceptible to contain said antibodies, characterized in that the saliva sample undergoes a pre-treatment, then said antibodies are detected in the pre-treated saliva by means of an immunoreaction with transglutaminase under conditions suitable to the formation of immuno-complexes with said antibodies. According to a preferred embodiment of the invention, the method is characterized in that it comprises the collection of salivas in sampling devices such as disclosed in the documents WO95/02996, U.S. Pat. No. 5,260,031, such as Omni-SAL® salivettes, subsequently the pre-treatment which comprises bringing the saliva in contact with N-acetyl-cystein.

The present invention also relates to a diagnostic kit for the diagnostic or the therapeutic monitoring of the coeliac disease. The present invention also relates to a method for screening and/or detecting and/or monitoring the coeliac disease in an individual, said method comprising the detection of anti-transglutaminase antibodies in a saliva sample from the individual. The present invention relates to a method which is able to facilitate the diagnostic of a coeliac disease and the monitoring of the disease evolution, without having to use blood extraction.

Thus, the present invention has an ability both for screening and monitoring the evolution in patients with coeliac disease under a gluten free diet. Moreover, the invention allows the diagnostic of all diseases associated with an immune reaction against transglutaminase or antigenic structures or related analogs thereof.

### Example 1

### 1. Materials and Methods

Samplings

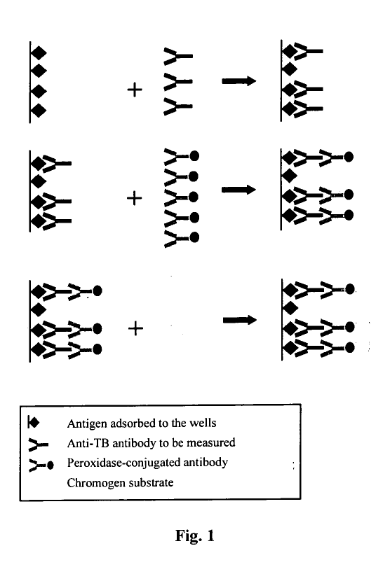

The tests are performed on saliva samples, collected by using Omni-SAL® salivettes. The collecting pad of the salivette is placed under the tongue until the indicator turns blue. The impregnated pad is then put back inside a tube containing a conservation liquid buffered to neutral pH and the saliva is extracted with the help of a filter of the piston type. The saliva is aliquoted and frozen to −80° C. The anti-transglutaminase ELISA concept The anti-transglutaminase ELISA concept is schematically depicted in FIG. 1.

The transglutaminase antigen either, purified from Guinea pig kidney (a proprietary process), or human recombinant (Celikey™ PHARMACIA Diagnostics) was adsorbed onto the wells of a microtitration plate. During a first incubation, the anti-transglutaminase antibodies possibly present within the samples are bound to the transglutaminase in the solid phase. After washing, a second incubation allows a peroxidase labeled human anti-IgA conjugate to bind to the anti-transglutaminase antibodies previously immobilized. A wash suppresses the conjugate in excess. In the presence of the chromogen substrate, the antigen-antibody-conjugate complexes are made apparent by means of a stained enzymatic reaction. The staining intensity, such as measured with a spectrophotometer, is reported on a standard curve, thus allowing an evaluation of the anti-transglutaminase antibody concentration.

ELISA Using Transglutaminase Purified from Guinea Pig Kidney According to an Embodiment of the Invention.

Material, Apparatus and Reactive Agents

The apparatus used for the assays comprise: an ELISA plate reading device, a PC with a processing software dedicated to the ELISA reading device (KC4V3.1), a refrigerator at 2-8° C., a freezer at −25° C., an oven at 37° C., a vortex, a balance, a pH meter.

The light equipment used includes: an ELISA plate (Nalge Nunc International) a Nunc IMMUNO Plate Brand Products Maxisorb surface, micropipettes from 0 to 10, 10 to 40, 50 to 200, 200 to 1000 μl, micropipettes with multichannels (8 channels) from 50 to 200 μl, sucking and emptying devices for pipettes from 2 to 10 ml, single use pipettes from 2 to 10 ml, plastic tubes with conic bottom from 5 to 10 ml, microtubes.

The commercial reactive agents used in the course of these assays include: Tris HCl, NaCl, CaCl₂, HCl 5N, EDTA (Kestranal A), Tween 20, dihydrated sodium citrate, distilled H₂O, purified transglutaminase from Guinea pig kidney (Sigma T5398), Standard anti-transglutaminase IgA at 10 000 AU ½ dilution in ethylene glycol →5 000 AU, 99% ethylene glycol (14.675.28 Janssen Chimica), anti-IgA human-peroxidase conjugate (HRP anti-IgA) from rabbit (Dako P216), OPD (Sigma P6912), 30% H₂O₂.

The following buffers were used:

Coating Buffer, pH 7.5: CaCl₂ (0.7 g), brought to 1 liter with distillated H₂O and adjusted to pH 7.5 with HCl 5N.

Dilution buffer Tris pH 7.4: Tris HCl (6.06 g), NaCl (8.8 g), EDTA (Kestranal A) (2.9 g), brought to 1 liter with distillated H₂O, added with 1 ml of Tween 20, and adjusted to pH 7.4 with HCl 5N.

Citrate buffer of the OPD substrate pH 4.2: C₆H₅Na₃O₇.2H₂O (29.4 g), brought to 1 liter with distillated H₂O and adjusted to pH 4.2 with HCl 5N.

Procedure

Plates Coating

100 μl of a transglutaminase solution with 10 μg/ml of a dilution buffer were distributed by well, the plate was covered up and incubated overnight at 4° C. Thereafter, the plate was washed 3 times with the dilution buffer.

100 μl of dilution buffer were distributed by well and the plate was incubated for 2 hours at 37° C.

Samples, Standard Curve and Blank Deposits

Standard dilutions in the dilution buffer: 1/100 dilution (100.00 AU) 1/400 dilution (25.00 AU) 1/800 Dilution (12.50 AU) 1/1600 dilution (6.25 AU) 1/6400 dilution (1.56 AU). The concentration range is from 1.56 AU to 100 AU.

The blank is constituted of the dilution buffer.

100 μl for each point of the curve ( 1/100, 1/400, 1/800, 1/1600, 1/6400), samples (undiluted saliva) and blank were distributed for each well, then incubated for 2 h at room T°, and washed 3 times with the dilution buffer.

Detection and Development

The conjugate was prepared on the basis of a 1/1000 dilution of the conjugate in the dilution buffer. 100 μl of such solution were distributed for each well, then incubated for 1 hour at room T°, and washed 3 times with the dilution buffer.

The substrate was prepared on the basis of 20 ml of a 1 mg/ml OPD solution in the citrate buffer and 40 μl of H₂O₂ were added, 200 μl of such solution were distributed for each well. The coloration is let to develop during 30 minutes at room T° away from direct light.

Reading: the absorbance was measured at 450 nm.

ELISA using human recombinant transglutaminase (Celikey™ Pharmacia Diagnostics)

Material, Apparatus and Reactive Agents

The apparatus used for these assays comprise: an ELISA plate reading device, a PC with a processing software dedicated to the ELISA reading device (KC4V3.1), a refrigerator at 2-8° C., a vortex.

The small equipment used comprises: microtitration wells coated with human recombinant transglutaminase, micropipettes from 0 to 10, 10 to 40, 50 to 200, 200 to 1000 μl, micropipettes with multichannels (8 channels) from 50 to 200 μl, sucking and emptying devices for pipettes from 2 to 10 ml, single use pipettes from 2 to 10 ml, plastic tubes with a conic bottom from 5 to 10 ml, microtubes.

The reactive agents used in the course of such assays include: 6 anti-transglutaminase antibody calibrators at 0-3-7-16-40-100 U/ml concentrations in BSA-containing PBS, 0.095% sodium azide (w/v), detergent and human serum, 1 ml each, ready for use.

The positive control was constituted of a BSA-containing buffer, 0.095% sodium azide (w/v), detergent and human serum, 1 ml each, ready for use.

The negative control was constituted of a BSA-containing buffer, 0.095% sodium azide (w/v), detergent and human serum, 1 ml, ready for use.

The concentrated washing buffer was constituted of 20× concentrated PBS containing 0.095% sodium azide (w/v) and detergent, 50 ml.

The IgA HRP conjugate was constituted of human anti-IgA antibodies HRP conjugate, 15 ml, ready for use.

Substrate TMB ready for use 15 ml.

The blocking solution was composed of H₂SO₄, 0.5M, 10 ml, ready for use.

Procedure

The wells were rinsed once with a washing buffer immediately before use, subsequently 100 μl of calibrators, control and samples (undiluted saliva) were distributed for each well. The Elisa plates were incubated during 30 minutes at room T°, and washed 3 times with a washing buffer. 100 μl of a conjugate solution were distributed for each well, subsequently incubated during 30 minutes at room T° and washed 3 times with the washing buffer. 100 μl of a substrate solution were then distributed for each well, subsequently incubated 10 min away from direct light at room T°. 50 μl of a blocking solution are ultimately distributed for each well and the absorbance was measured at 450 nm between 10 and 30 minutes after the addition of the blocking solution.

### 2. Results

2.1 Adjustment of the Anti-Transglutaminase ELISA on Salivary Samplings According to One of the Embodiment of the Present Invention.

The first assays were performed using, stricto sensu, the ELISA protocol disclosed hereunder.

The IgA total concentrations in the serum (75 to 400 mg/100 ml) were about 10 times higher than in the saliva (9 to 32 mg/100 ml). The results obtained were therefore compared by testing the saliva as such (1/1 diluted) with the 10 times diluted saliva, the latter dilution being thus equivalent to the 1/100 dilution of a serum sample such as recommended in the initial process. Four individuals with the coeliac disease (C) and four control individuals (N) were tested. It appeared (Table 1) that the 1/10 dilution of saliva decreased too much the sensitivity of the test since the OD, in particular those of the patients with the coeliac disease slumped. The following assays were conducted with the 1/1 dilution (pure saliva).

TABLE 1
Comparison between 1/1 (not diluted) and 1/10 saliva dilutions.

OD

| | SAMPLES | Dil 1/1 | Dil 1/10 |
|---|---|---|---|
| N | OCM | 0.252 | 0.143 |
| N | HAS | 0.195 | 0.152 |
| N | NAJ | 0.233 | 0.061 |
| N | SOP | 0.159 | 0.04 |
| C | DEC | 0.146 | 0.025 |
| C | BOU | 0.332 | 0.064 |
| C | NOE | 0.484 | 0.115 |
| C | VIL | 0.405 | 0.118 |

The various steps of the protocol were examined in order to find out the optimum conditions both in terms of sensitivity and specificity.

Pre-Treatment of Saliva With NAC

NAC (N-acetyl-cystein) is a reducing agent known to decrease the viscosity of the bronchial secretions. The mucins, glycoproteins present in the saliva, can on one hand bind the IgAs and on another hand form a deposit on the microtitration plates and therefore are responsible of the background noise observed for the negative samples.

In a first step, the required NAC optimum concentration was determined.

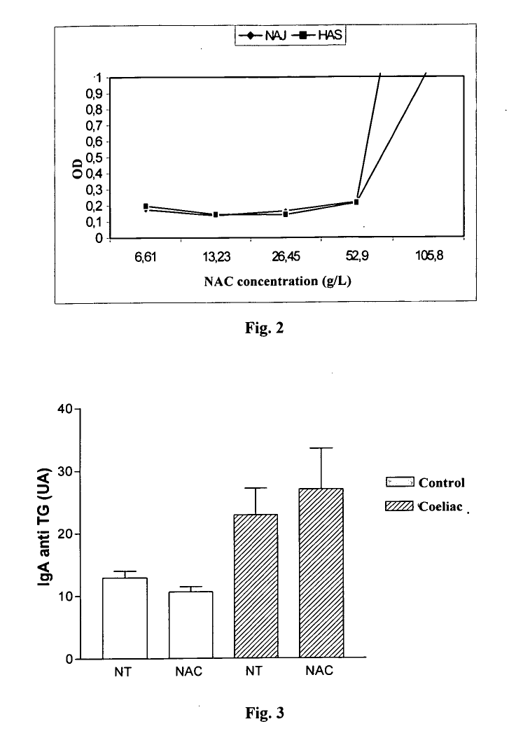

The determination results of the NAC optimum concentration are shown in Table 2 and FIG. 2.

TABLE 2
Evaluation of the NAC optimum concentration

| | | OD | | | | | |
|---|---|---|---|---|---|---|---|
| | | | Treated with NAC | | | | |
| | Samples | not treated | 105.8 g/L | 52.90 g/L | 26.45 g/L | 13.23 g/L | 6.61 g/L |
| N | NAJ | 0.233 | 2.663 | 0.219 | 0.166 | 0.138 | 0.173 |
| N | HAS | 0.213 | 1.049 | 0.214 | 0.143 | 0.145 | 0.196 |

In a preferred embodiment of the invention the optimum concentration is 13.23 g/L for the dilution buffer, as a matter of fact, it is at this concentration that the OD of the controls are the lowest.

A Comparison from Treated with Non Treated Salivas

5 μl of a NAC solution (Sigma Grade A7250) at 13.23) g/L in the dilution buffer for 100 μl of saliva were used in order to obtain an final concentration of 0.625 g/L of saliva, the samples incubation in the presence of NAC was made at room T° during 20 minutes.

The results obtained are shown in Table 3 and FIG. 3

TABLE 3
A comparison between salivas both non treated and treated with NAC

| | | TRIAL 1 (OD) | | TRIAL 2 (OD) | | TRIAL 3 (OD) | | TRIAL 4 (OD) | |
|---|---|---|---|---|---|---|---|---|---|
| | | Non | | Non | | Non | | Non | |
| | SAMPLES | treated | treated | treated | treated | treated | treated | treated | treated |
| N | NAJ | 0.233 | 0.157 | 0.286 | 0.228 | 0.263 | 0.222 | | |
| N | HAS | 0.213 | 0.164 | 0.340 | 0.277 | | | 0.282 | 0.273 |
| N | ING | | | 0.377 | 0.255 | 0.470 | 0.404 | | |
| N | SOP | | | | | 0.391 | 0.325 | | |
| N | VIR | | | | | 0.429 | 0.328 | | |
| N | OCM | | | | | 0.406 | 0.376 | | |
| N | STE | | | | | | | 0.421 | 0.421 |
| N | J.MI | | | | | | | 0.561 | 0.529 |
| N | M.R | | | | | | | 0.421 | 0.398 |
| N | CAR | | | | | | | 0.280 | 0.317 |
| C | NOE | | | 0.347 | 0.370 | | | 0.413 | 0.376 |
| C | VIL | | | | | 1.100 | 0.612 | 0.907 | 0.703 |
| C | BOU | | | | | 0.844 | 0.897 | 0.562 | 0.657 |
| C | DEW | | | | | | | 1.210 | 1.645 |
| C | DEC | | | | | | | 0.364 | 0.277 |
| C | EXT | | | | | | | 0.585 | 0.748 |
| C | DEV | | | | | | | 0.739 | 1.259 |
| C | ZBI | | | | | | | 0.762 | 1.557 |
| C | COO | | | | | | | 0.073 | 0.159 |
| C | FON | | | | | | | 0.586 | 0.746 |
| C | GRE | | | | | | | 0.271 | 0.272 |

The pre-treatment with NAC of the salivas allowed to decrease the negative control signal and to increase that of the patients with coeliac disease; such surprising effect led to an improved detection thereof and to a more extended discrimination between both populations.

In order to demonstrate the effect of NAC, meaning the transformation of the IgA secretory dimers into monomers, a radial immunodiffusion was performed. The saliva was deposited on a gel containing antibodies that exclusively recognize the secretory IgA. After 5 days of incubation, a precipitation halo was observed for the salivas not treated with the NAC corresponding to the reaction of the secretory IgA present in the saliva with the antibodies included in the gel. In contrast, for the NAC treated salivas, the halo is not observed which is a sign of the absence of secretory IgAs.

2.2 Comparison of the ELISA Using of Either a Purified Transglutaminase from Guinea Pig Kidney (TG), or a Human Recombinant Transglutaminase (Celikey™ Pharmacia Diagnostics) (Celikey TG)

The benefits drawn from pre-treating the samples with the NAC was evaluated on the Celikey, which used the human recombinant transglutaminase. It is now well admitted, as to the serum samples, that the ELISAs using human recombinant TG are more specific than those using Guinea pig TG.

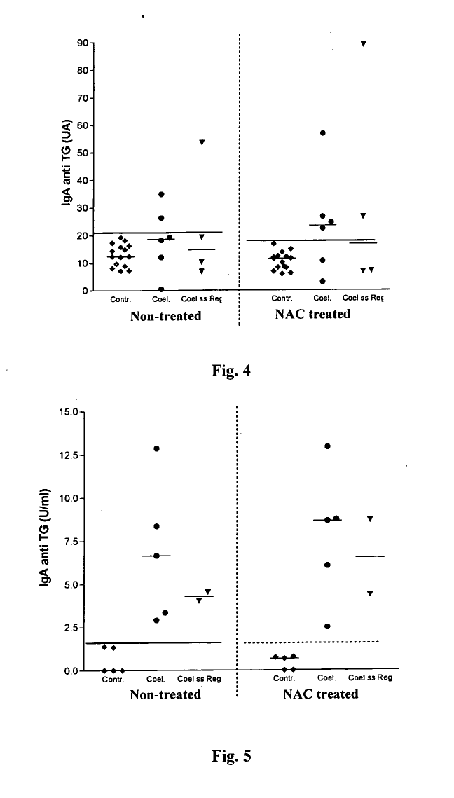

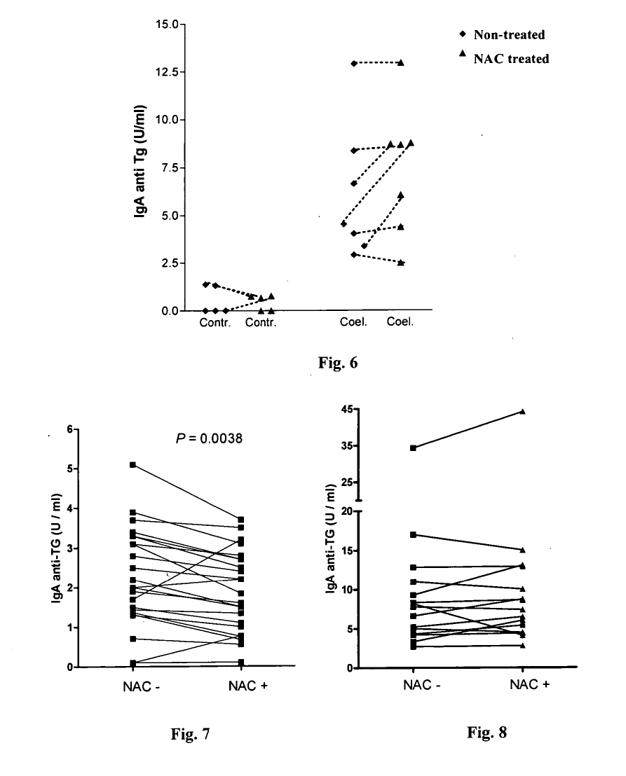

The results obtained are shown in Table 4 and FIGS. 4 (antitG IgA), 5 (Celikey antitG IgA) and 6.

TABLE 4
Table giving a summary of the results for blood and saliva

| | | | | SALIVA | | | |
|---|---|---|---|---|---|---|---|
| | | | | | | TG | TG NAC |
| | | BLOOD | | | TG | (Celikey) | (Celikey) |
| | Samples | Endomysium | TG(AU) | TG(AU) | NAC(AU) | (U/ml | (U/ml) |
| Threshold | | | | 21 | 17 | 1.6 | 1.6 |
| value | | | | | | | |
| N | NAJ | | | 9.7 | 8.22 | | |
| N | HAS | | | 7.2 | 6.88 | 0 | 0 |
| N | ING | | | 17.34 | 14.85 | | |
| N | OCM | | | 14.93 | 13.81 | | |
| N | VIR | | | 15.79 | 12.05 | | |
| N | J.MI | | | 18.15 | 16.79 | 1.32 | 0.67 |
| N | SOP | | | 14.37 | 11.94 | 1.37 | 0.75 |
| N | STE | | | 12.4 | 12.4 | 0 | 0 |
| N | M.R | | | 12.4 | 11.5 | 0 | 0.77 |
| N | CAR | | | 7.12 | 8.46 | | |
| C | NOE | 1/800 | >120 | 12.09 | 10.66 | | |
| C | COO | | | <1.27 | 3.01 | | |
| C | VIL | 1/200 | 51 | 34.93 | 24.56 | 2.89 | 2.47 |
| C | BOU | 1/200 | >100 | 18.19 | 22.42 | 6.63 | 8.75 |
| C | EXT | 1/100 | >120 | 19.19 | 26.73 | 8.34 | 8.65 |
| C | DEV | | | 26.29 | 56.82 | 3.34 | 6.04 |
| C | GLO | 1/400 | >105 | | | 12.86 | 12.92 |
| SR | FON | neg | neg | 19.23 | 26.63 | 4.01 | 4.35 |
| SR | DEC | 1/400 | 62 | 10.207 | 7.02 | 4.51 | 8.68 |
| SR | GRE | 1/200 | nd | 6.8 | 6.84 | 0 | |
| SR | DEW | neg | neg | 53.42 | 88.97 | 14.23 | |

The NAC treatment of the salivas leads to a signal decrease of the controls (see FIG. 4): the anti-TG IgA concentration median changes from 12.4 AU to 11.5 AU after treatment. In contrast, there is an increase of the signal from patients with coeliac disease, the concentration median changing from 18.7 AU to 23.5 AU. The threshold values (an average of 5 controls+2DS), are 21 AU with no treatment and 17 AU with the treatment.

Consequently, the test sensitivity and specificity with the TG from guinea pig kidney are 30% and 100% respectively with no NAC treatment and 60% and 100% with the treatment.

Two patients with coeliac disease not being under a diet were not detected, even after the salivary samples had been treated with the NAC. For one of them, the serum tests being positive with regard to anti-TG let, a response equally positive on the saliva was expected. For these 2 patients, using an immunodiffusion technique, the secretory IgA in the non treated saliva were not detected, which demonstrate a probable degradation of the salivary samples. Nevertheless, also by immunodiffusion, it was demonstrated that those still contained IgA. Knowing that these samplings had undergone several freezing/unfreezing rounds, a degradation is then most likely to occur and would explain the negative results for these patients.

Additionally, the surprising effect of the NAC based pre-treatment was confirmed for the Celikey test.

The increase of the control median after treatment was due to the results of a single sample (MR) that gave a response slightly higher after treatment (0 U/ml and 0.77 U/ml).

The NAC based pre-treatment of the saliva allowed to increase the signal of the patients with coeliac disease since, out of 7 patients, 6 showed an increase of their signal with the NAC (see FIG. 5). Here, the threshold value (1.6 U/ml) was calculated from a preliminary trial conducted on a more extended series of samples. Such threshold value was also used for the NAC treated salivas.

As a conclusion, an ELISA test was implemented for the screening of anti-transglutaminase (TG) IgA in salivary samplings. The assays conducted by using the protocol for anti-TG IgA screening in the serum, show little discrimination between the results from the control individuals and the patients with coeliac disease.

Hence, on of the objectives was to decrease the signal from the controls. The advantage of a saturation with BSA or casein was evaluated and the uselessness of a saturation step was demonstrated. Various concentrations (1 ml/L and 50 ml/L) of Tween 20 were compared using as a dilution buffer either Tris or PBS. The decomplementation proved inappropriate, indeed, it induces a drop of the positive controls signal.

The N-acetyl-cystein (NAC) reducing power allowed to transform the secretory IgA (dimeric) into monomers and thus to improve the response from the patients with coeliac disease.

Furthermore, this agent breaks the IgA-mucins bonds responsible of non specific bindings. After an NAC based treatment, a surprising discrimination between both populations is observed, the signal from the controls decreasing and the response from the patients with coeliac disease increasing after treatment of the saliva.

Moreover, the advantage of the NAC based treatment of the salivas was confirmed using the Celikey, anti-transglutaminase ELISA using as an antigen the human recombinant TG. The present invention allows the screening of the coeliac disease using salivary samplings.

### Example 2

### Confirmation of the Positive Effect of the NAC Based Treatment on a Larger Number of Samples

The discrimination between patients with coeliac disease and healthy control subjects was evaluated:
according to whether the saliva samples were or not NAC treated
by testing the salivas from 15 non-treated patients with coeliac disease and from healthy control 25 subjects
by collecting all the salivas on salivette.

FIGS. 7 and 8 represent graphs comparing the level of anti-TG IgA in both non-treated and treated with N-acetyl-cystein salivas from healthy control subjects (FIG. 7) and non-treated patients with coeliac disease (FIG. 8). The results illustrated in FIGS. 7 and 8 show that the effect of NAC is very important on the control salivas, decreasing in a significant manner (P=0.0038) the values obtained for the control salivas. This allows to decrease the value selected as a “cut-off” value for determining that a saliva sample contains anti-TG IgAs. As a matter of fact, if a 100% specificity corresponding to the totality of the negative control salivas is wished, the “cut-off” value must be fixed above 5.1 U in the absence of a NAC based treatment and it can be decreased to 3.7 U upon a NAC based treatment.

Sensitivity and Specificity of the Anti-TG IgAs Detection in the Saliva for the Diagnostic of Non-Treated Coeliac Disease.

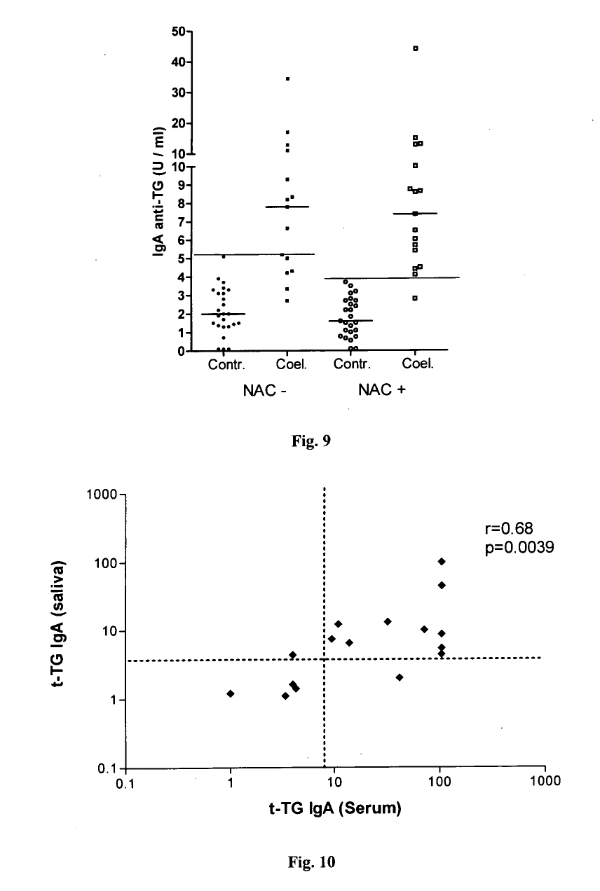

The sensitivity and specificity of the anti-TG IgA detection in the saliva for the diagnosis of non treated patients with coeliac disease are shown in Table 5 and FIG. 9. FIG. 9 represents a graph comparing the level of anti-TG IgA in non treated salivas and salivas treated with N-acetyl-cystein in healthy control subjects and in non treated patients with coeliac disease, the cut-off values being represented by the thin lines on the graph.

TABLE 5
Sensitivity and specificity of the anti-TG IgAs detection
in the saliva for the diagnostic of the non treated
patients with coeliac disease

| | NAC− | NAC+ |
|---|---|---|
| Cut-off | 5.2 U/ml | 4 U/ml |
| Specificity | 100% | 100% |
| Sensitivity | 60% | 94.12% |

As a conclusion. The salivary test for the anti-TG IgAs dosing in the saliva as developed herein offers for the diagnostic of non treated patients with coeliac disease a sensitivity that can be compared to that disclosed in the literature for serum tests and this with a 100% specificity.

Furthermore, the salivary results were compared to the serum results for 16 individuals. The results obtained and shown in FIG. 10 demonstrate an excellent correlation (Spearman's test of correlation: r=0.68, p=0.0039). The samples considered in that graph were collected from patients with coeliac disease either before initiation of a diet, or in the course of treatment. The inclusion criterion is a time limit of less than a month between the saliva collection and that of the serum. Hence, these samples are not necessarily the same as those reported in the graph of FIG. 9.

Diagnostic Utility of the Developed Test Among a Population of Patients

In the above paragraph, the specificity and sensitivity of the test developed for the diagnostic of the coeliac disease were calculated by comparing the results obtained for patients with coeliac disease to those obtained for healthy individuals as is usually done.

Here, were tested saliva samples collected from 37 patients consulting at the gastroenterology clinic department suffering from clinical pains compatible with those of the patients with coeliac disease. For 17 of them, the diagnostic of the coeliac disease was seriously contemplated and confirmed by the results of an intestine biopsy. For the twenty others, the diagnostic of the coeliac disease was rejected by the clinician on the basis of an intestine biopsy which was not compatible with a diagnostic of coeliac disease.

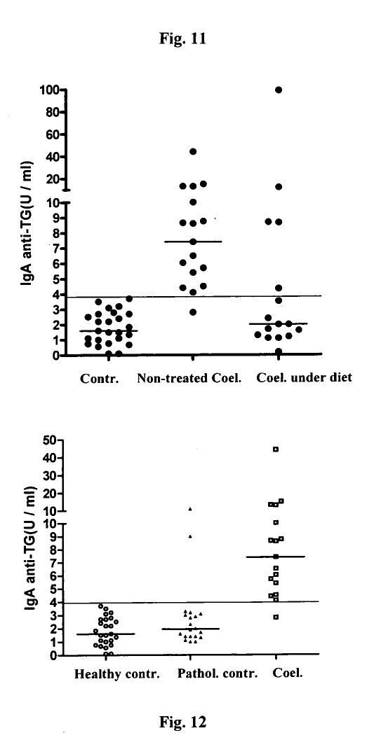

Upon a comparison between the results obtained for both groups of patients, there again an excellent discrimination is obtained on the basis of the anti-TG IgAs detected in the saliva. The results obtained with the healthy controls and the “pathologic” controls were not significantly different. Only two patients considered as patients not having coeliac disease were positive. The results of such comparison are shown in FIG. 11.

An Evaluation of the Test in Patients with Coeliac Disease Who Follow a Gluten Free Diet

As soon as the diagnostic of coeliac disease is made, the patients should follow a strict gluten free diet and the anti-transglutaminase IgAs become no more detectable in the serum, after a variable delay according to the individuals and to the compliance with the diet.

The anti-TG IgAs in the saliva of 16 patients with coeliac disease under a diet were also measured. The results depicted in FIG. 12 show that most of the salivas became negative so that this test can be used also for the monitoring (control) of gluten free diet.

What is claimed is:

1. A method for detecting and/or dosing anti-transglutaminase antibodies in a saliva sample susceptible to contain said antibodies, characterized in that the saliva sample is subjected to a pre-treatment, then in that said antibodies are detected in the pre-treated saliva by an immune reaction with transglutaminase under conditions suitable to the formation of immuno-complexes with said antibodies.

2. The method according to claim 1, characterized in that the method comprises the collection of the salivas in a sampling device, then the pre-treatment which comprises bringing the saliva in contact with a mucolytic compound.

3. The method according to claim 2, characterized in that the saliva sampling device comprises: a sample collector which selectively receives a saliva sample; and an indicator activated by a preselected amount of the received saliva sample in order to indicate that the amount of the collected biological fluid sample is convenient.

4. The method according to claim 3, characterized in that the sampling device is selected from the group comprising: Omni-SAL® (Saliva The diagnostic Systems, Inc . . . ), Salivette® (Sarstedt Ltd.), Orapette® (Trinity Biotech) and OraSure® (Epitope, Inc.), preferably Omni-Sal®.

5. The method according to any claims 2 to 4, characterized in that the mucolytic compound is selected from the group comprising N-acetyl-cystein, nacystelyn, dithiothreitol, gelsolin, thioredoxin et EDTA.

6. The method according to any claims 2 to 5, characterized in that the mucolytic compound is N-acetyl-cystein.

7. The method according to of any claims 1 to 6, characterized in that the transglutaminase is from human, animal, synthetic or recombinant origin.

8. The method according to any claims 1 to 7, characterized in that the detection is performed in an immunoassay, preferably by direct or non direct coupling to a reaction partner having a labeling substance easy to detect.

9. The method according to claim 8, characterized in that the detection is preformed in an ELISA test.

10. The method according to any claims 1 to 9, characterized in that the immunocomplexes formed are brought in contact with a conjugate comprised of a labeled antibody directed against said antibodies under conditions suitable to the formation of the labeled immuno-complexes, then the labeled immunocomplexes are detected and quantified.

11. The method according to claim 10, characterized in that the conjugate is an anti-immunoglobulin antibody labeled with an alkaline phosphatase or with a peroxidase, and in that the formation of labeled immuno-complexes is detected and quantified by calorimetric or fluorimetric measurement.

12. Use of the method according to any claims 1 to 11 for the detection of gluten induced diseases.

13. Use of the method according to claim 12, characterized in that the disease to be detected is the coeliac disease.

14. Use of the method according to any claims 1 to 11 for the monitoring of a gluten free diet.

15. The method for the diagnostic or therapeutic monitoring of the coeliac disease, characterized in that anti-transglutaminase antibodies are detected in a saliva sample susceptible to contain said antibodies, characterized in that the saliva sample is subjected to a pre-treatment, and in that said antibodies are detected in the pre-treated saliva by an immune reaction with transglutaminase under conditions suitable to the formation of immuno-complexes with said antibodies.

16. The method for the diagnostic or monitoring according to claim 15, characterized in that the method comprises the collection of the saliva in a sampling device, and the pre-treatment which comprises bringing the saliva in contact with a mucolytic compound.

17. The method for the diagnostic or monitoring according to claim 16, characterized in that the saliva sampling device comprises: a sample collector which selectively receives a saliva sample; and an indicator activated by a preselected amount of the received saliva sample in order to indicate that the amount of the collected biological fluid sample is convenient.

18. The method for diagnostic or monitoring according to claim 17, characterized in that the sampling device is selected from the group comprising: Omni-SAL® (Saliva The diagnostic Systems, Inc . . . ), Salivette® (Sarstedt Ltd.), Orapette® (Trinity Biotech) and OraSure® (Epitope, Inc.), preferably Omni-Sal®.

19. The method for diagnostic or monitoring according to any claims 16 to 18, characterized in that the mucolytic compound is selected from the group comprising N-acetyl-cystein, nacystelyn, dithiothreitol, gelsolin, thioredoxin and EDTA.

20. The method for diagnostic or monitoring according to any claims 16 to 19, characterized in that the mucolytic compound is N-acetyl-cystein.

21. A diagnostic kit for diagnostic or therapeutic monitoring of the coeliac disease according to any claims 15 to 20.
